(19) United States
(12) Patent Application Publication
(10) Pub. No.: US 2017/0107510 A1
(43) Pub. Date: Apr. 20, 2017

(54) DNA NANOPARTICLE BASED CHECKPOINT INHIBITORS

(71) Applicant: Batu Biologics, Inc., San Diego, CA (US)

(72) Inventors: Samuel C. Wagner, San Diego, CA (US); Thomas E. Ichim, San Diego, CA (US)

(21) Appl. No.: 15/293,999

(22) Filed: Oct. 14, 2016

Related U.S. Application Data
(60) Provisional application No. 62/241,561, filed on Oct. 14, 2015.

(51) Int. Cl. C12N 15/10; C12N 15/115

(52) U.S. Cl. CPC C12N 15/1093; C12N 15/115; C12N 2310/16

(57) ABSTRACT

The invention discloses the utilization of DNA generated nanoparticles to block interaction between inhibitory signals in immune cells. Provided are compositions of matter, protocols and treatment methodologies for stimulation immunity through inhibiting suppressive molecules through the use of DNA based nanoparticles.

### CROSS REFERENCE TO RELATED APPLICATION

This application claims priority to U.S. Provisional Patent Application 62/241,561 filed on Oct. 14, 2015, the entirety of which is incorporated herein by reference.

### BACKGROUND

The immune system is comprised of activatory and inhibitory mechanisms which allow for tight control of immune responses and subsequent inhibition of responses after clearance of the immune target. At a very basic level, one may consider that the central event stimulating immune responses is the antigen-specific activation of naive CD4⁺ T cells subsequent to binding antigen presenting cell MHC containing antigenic peptide. The CD4+ T cell, also known as the “helper T cell” coordinates the activation of the adaptive immune response, being critical for stimulation of cytotoxic CD8+ T cells, whose role is to destroy host cells affected by cancer, viruses, and intracellular bacteria, as well as to stimulate B cell maturation to eventual plasma cell differentiation, which is responsible for antibody production. Antibodies being critical molecules in clearance of extracellular pathogens such as various bacteria and parasites. It is generally accepted that, under most circumstances, naive CD4+ T cells require two distinct signals to proliferate and differentiate into the armed effector cells that mediate adaptive immunity. Signal 1 of this two-signal model is antigen-specific and is generated by interaction of the TCR with antigenic peptide presented in context with MHC II antigens. This results in transduction of TCR intracellular signals leading to production of IL-2 and T cell activation. Signal 2 is referred to as a costimulatory signal because, while essential, it does not by itself induce any functional response in T cells. The best characterized costimulatory signal 2 is generated through the T cell surface molecule CD28. CD28 delivers a costimulatory signal upon interaction with CD80 and/or CD86) present on B cells, macrophage, or dendritic cells. Activation of the TCR in the presence of costimulatory signals leads to T cell clonal expansion and initiation of effector functions such as IL-2 production.

In the situation of cancer, immune inhibitory mechanisms, termed immune checkpoints, are prematurely activated in order for the tumor to escape immune attack. Two main immune checkpoints exist: a) CTLA-4, which sends an inhibitory signal to T cells upon binding CD80 and/or CD86 on antigen presenting cells, and; b) PD-1, which binds to PD-1 ligand on tumor cells, stromal cells, or antigen presenting cells.

CTLA-4 is related to CD28, however instead of activating T cells in a co-stimulatory manner, it leads to inhibition or co-inhibition of T cells.

### SUMMARY OF THE INVENTION

The following presents a simplified overview of the example embodiments in order to provide a basic understanding of some aspects of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented.

The invention discloses a means of generating DNA-based nanoparticles capable of binding and selectively blocking function of immune inhibitory molecules. Said immune inhibitory molecules are widely known in the art, in some contexts, as “checkpoint inhibitors”. Currently great successes have been achieved by modulating checkpoint inhibitor molecules such as PD-1, PD-1 ligand, and CTLA-4. Unfortunately, the only therapeutic means of intervening on the signaling of said checkpoint inhibitors has been through utilization of antibody mediated blockade. Antibodies possess numerous drawbacks including immunogenicity, need for constant administration, and poor tumor penetration. The current invention teaches the use of DNA-based nanoparticles as effective blocking agents of checkpoint inhibitors.

### DETAILED DESCRIPTION OF THE PREFERRED EMBODIMENTS

The invention discloses a means of generating DNA-based nanoparticles capable of binding and selectively blocking function of immune inhibitory molecules. Said immune inhibitory molecules are widely known in the art, in some contexts, as “checkpoint inhibitors”. Currently great successes have been achieved by modulating checkpoint inhibitor molecules such as PD-1, PD-1 ligand, and CTLA-4. Unfortunately, the only therapeutic means of intervening on the signaling of said checkpoint inhibitors has been through utilization of antibody mediated blockade. Antibodies possess numerous drawbacks including immunogenicity, need for constant administration, and poor tumor penetration. The current invention teaches the use of DNA-based nanoparticles as effective blocking agents of checkpoint inhibitors.

In the practice of the invention, replication of DNA that is circular in form is achieved using a strand displacing polymerase to generate long linear concatemers of controllable length that generated in a manner allowing for spontaneous folding into a ball conformation due to internal base-pairing. These DNA based nanoparticles can be generated as particles of distinct size based on the amount of cycles and conditions of generation. For practice of the invention, sizes of nanoparticles generated are of sufficient size to achieve pharmacokinetic distribution into the extracellular fluid compartment. Preferable sizes for generated nanoparticles are between 2-200 nanometers, more preferably between 5-50 nanometers, more preferable between 10-30 nanometers. Said DNA nanoparticles are screened for binding to immune inhibitory molecules such as checkpoint inhibitors, DNA nanoparticles binding to said immune inhibitory molecules are subsequently selected, amplified and utilized as inhibitors for in vivo use and clinical development.

In one embodiment of the invention DNA nanoparticles are generated by contacting a circular single-stranded nucleic acid template with a nucleic acid polymerase, wherein the nucleic acid template encodes an aptamer. Subsequently said template is amplified with said polymerase to produce said nanoparticle, wherein said nanoparticles comprises a concatemer of the sequence of said template. In one embodiment, the nucleic acid polymerase is a strand displacing polymerase, such as a DNA polymerase, and can be selected from the group consisting of phi29 polymerase, Klenow fragment, Bst DNA polymerase, M-MuLV reverse transcriptase, and AMV reverse transcriptase. In some embodiments, the amplifying step has a duration of more than about 1, 5, 10, 25, 30, 50, and 120 minutes. Some methods of making a nanoparticle can also include circularizing a linear nucleic acid template to produce the circular nucleic acid template. The linear nucleic acid template can be more than 10, 50, 100, or 1000 bases in length.

In one embodiment nanoparticles are generated from DNA that can be more than 1 kb, 10 kb, 100 kb, 1 Mb, 10 Mb, 100 Mb, and 500 Mb in length. Specific targeting of DNA nanoparticles can be achieved by identifying nanoparticles containing aptamers including generating a library of nanoparticles comprising putative aptamers; and screening the library. The screening is performed by assessing binding to immune inhibitory molecules. Said immune inhibitory molecules may be plate bound, or otherwise conjugated to a solid surface.

Checkpoint inhibitory nanoparticles can be produced in a scalable manner utilizing available techniques in the art. Said nanoparticle production begins with a padlock probe of single stranded DNA. The sequence of this probe is engineered for the specific purpose of blocking proteins possessing immune inhibitory activities, said probe can vary in length from at least about 2 bases, at least about 5 bases, at least about 10 bases, at least about 50 bases, at least about 100 bases, at least about 500 bases, at least about 750 bases, at least about 1000 bases, at least about 1 kb, at least about 5 kb, at least about 10 kb, at least about 50 kb, and longer. In one embodiment, said probe is ligated endwise to form a closed loop circle via a templating primer complementary to the ends of the probe. The templating primer then serves as a primer for polymerization with a strand displacing polymerase with the circle acting as an endless template in rolling circle amplification as described by Messmer's group. Specifically, it has been shown that rolling circle amplification of circularizable oligonucleotides, also termed “padlock” probes, is a sensitive method for the detection and amplification of short DNA sequences. Typically the term padlock probe is used to describe a single-stranded oligonucleotide whose 5′ and 3′ ends hybridize to a target of interest and are then ligated to create a single-stranded circular DNA molecule, which is then a substrate for rolling circle amplification. The sensitivity of DNA ligase to mismatches and the fact that the ligated probe will remain hybridized to the target when subjected to stringent washing conditions result in a very high signal-to-noise ratio. As such, padlock probes can be used for the detection of gene sequences and variants because the target can be detected directly. Since the regions of the probe not involved in target binding are arbitrary, they can be designed to encode hybridization targets for differentiable probes or other sequence moieties that facilitate simultaneous multiplexed analysis. Such strategies are generally less susceptible to cross-reactions than PCR. For the purpose of the invention, polymerization can proceed for a period of time, after which a certain length of single stranded DNA is created comprising concatamer repeats of the original padlock probe. In one specific embodiment of the invention, the concatamer can spontaneously form a globular shape based on internal base pairing. The size of the nanoparticles can be controlled by, for example, the polymerization time of the reaction, the type of polymerase used, and reaction conditions such as salt concentration and acidity. Some embodiments of the invention include nanoparticles with aptamers for use in blocking checkpoint inhibitors. In one embodiment of the invention, chemical libraries are utilized as source of potential ligands for inhibitors of immunological checkpoints. When modular biopolymers such as nucleic acids or polypeptides are used, the combinatorial diversity of these libraries can become astronomical and well beyond the capabilities of systemic high throughput screening methods. In the practice of the invention screening of the libraries then requires iterative schemes that couple a selection step with an amplification step. For peptides, display of a given peptide on a bacteriophage, virus, or bacteria allows amplification by growth of the host organism. Nucleic acids are typically amplified by some variation of PCR. These tools have been used to select peptides and short nucleic acid sequences (aptamers) that can bind to a wide variety of proteins and cellular targets.

In one embodiment of the invention, DNA nanoparticles are created to block the interaction between inhibitory ligands and inhibitory receptors. Said inhibitory receptors include immunological checkpoints. A means of overcoming immune suppression in cancer is by blocking inhibitory signals generated by the tumor, or generated by cells programmed by the tumor. In essence, all T cells possess costimulatory receptors, such as CD40, CD80 and CD86, which are also known as “signal 2”. In this context, Signal 1 is the MHC-antigen signal binding to the T cell receptor, whereas signal 2 provides a costimulatory signal to allow for the T cells to produce autocrine IL-2 and differentiate into effector and memory T cells. When T cells are activated in absence of signal 2 they become anergic or differentiate into Treg cells. The costimulatory signals exist as a failsafe mechanism to prevent unwanted activation of T cells in absence of inflammation. Indeed, most of the inflammatory conditions associated with pathogens are known to elicit signal 2. For example, viral infections activate toll like receptor (TLR)-3, 7, and 8. Activation of these receptors allows for maturation of plasmacytoid dendritic cells which on the one hand produce interferon alpha, which upregulates CD80 and CD86 on nearby cells, and more directly, the activation of these TLRs results in the plasmacytoid dendritic cell upregulating costimulatory signals. In the case of Gram negative bacteria, upregulation of signal 2 is mediated by LPS binding to TLR-4 which causes direct maturation of myeloid dendritic cells and thus expression of CD40, CD80 and CD86, as well as production of cytokines such as IL-12 and TNF-alpha, which stimulate nearby cells to upregulate signal 2.

Once immune responses have reached their peak, coinhibitory receptors start to become upregulated in order to suppress an immune response that has already performed its function. This is evidenced by upregulation of coinhibitory molecules on T cells such as CTLA4, PD-1, TIM-3, and LAG-3. The finding of co-inhibitory receptors has led to development of antibodies against these receptors, which by blocking their function allow for potent immune responses to ensure unrestrained. The advantage of inhibiting these “immunological checkpoints” is that they not only allow for T cell activation to continue and to not be inhibited by Treg cells, but they also allow for the T cell receptor to become more promiscuous. By this mechanism T cells start attacking various targets that they were not programmed initially to attack.

The currently approved checkpoint inhibitors, which block CTLA-4 and PD-1, great clinical progress has been achieved in comparison to previous treatments that were available. In the example of CTLA-4 inhibition ipilimumab has been approved by regulators and tremelimumab is in advanced stages of clinical trials. Although these anti-CTLA-4 antibodies have modest response rates in the range of 10%, ipilimumab significantly improves overall survival, with a subset of patients experiencing long-term survival benefit. In a phase III trial, tremelimumab was not associated with an improvement in overall survival. Across clinical trials, survival for ipilimumab-treated patients begins to separate from those patients treated in control arms at around 4-6 months, and improved survival rates are seen at 1, 2, and 3 years. Further, in aggregating data for patients treated with ipilimumab, it appears that there may be a plateau in survival at approximately 3 years. Thereafter, patients who remain alive at 3 years may experience a persistent long-term survival benefit, including some patients who have been followed for up to 10 years.

In the case of PD-1 inhibition, Herbst et al. evaluated the single-agent safety, activity and associated biomarkers of PD-L1 inhibition using the MPDL3280A, a humanized monoclonal anti-PD-L1 antibody administered by intravenous infusion every 3 weeks (q3w) to patients with locally advanced or metastatic solid tumors or leukemias. Across multiple cancer types, responses as per RECIST v1.1 were observed in patients with tumors expressing relatively high levels of PD-L1, particularly when PD-L1 was expressed by tumor-infiltrating immune cells. Specimens were scored as immunohistochemistry 0, 1, 2, or 3 if <1%, ≧1% but <5%, ≧5% but <10%, or ≧10% of cells per area were PD-L1 positive, respectively. In the 175 efficacy-evaluable patients, confirmed objective responses were observed in 32 of 175 (18%), 11 of 53 (21%), 11 of 43 (26%), 7 of 56 (13%) and 3 of 23 (13%) of patients with all tumor types, non-small cell lung cancer (NSCLC), melanoma, renal cell carcinoma and other tumors (including colorectal cancer, gastric cancer, and head and neck squamous cell carcinoma). Interestingly, a striking correlation of response to MPDL3280A treatment and tumor-infiltrating immune cell PD-L1 expression was observed. In summary, 83% of NSCLC patients with a tumor-infiltrating immune cell IHC score of 3 responded to treatment, whereas 43% of those with IHC 2 only achieved disease stabilization. In contrast, most progressing patients showed a lack of PD-L1 upregulation by either tumor cells or tumor-infiltrating immune cells.

In one embodiment of the invention chemical libraries are used to screen compounds that block or substantially inhibit immunological checkpoints, in the same manner that phage display is used, chemical libraries or libraries of DNA nanoparticles are utilized to generate inibitors. By utilizing biopolymers such as nucleic acids or polypeptides diversity of these libraries can become astronomical and well beyond the capabilities of systematic high throughput screening methods. Screening said libraries requires iterative schemes that couple a selection step with an amplification step. For peptides, display of a given peptide on a bacteriophage, virus, or bacteria allows amplification by growth of the host organism. In one embodiment selection of peptides for ability to bind and inhibit checkpoints of the immune system are disclosed. Nucleic acids are typically amplified by some variation of PCR. Subtractive and in vivo selection schemes have been developed for aptamer and phage displayed peptide libraries that can enhance the cell specificity of recovered targeting ligands. Most peptide display formats, such as phage, present many copies of each peptide per particle. This can allow the recovery of relatively low affinity interactions that benefit from the high avidity of the presentation format. However, it may be difficult to maintain the desired binding avidity and specificity when the selected peptides are moved to another particle or molecule. Aptamer libraries can suffer from the reverse complication since they are usually presented in a monovalent format. Aptamers have been most clinically useful when a high affinity interaction can function in an antagonist manner, though they have been used as targeting moieties attached to nanoparticle drug delivery vehicles. However, when aptamers that were selected in a monovalent format are attached to particles in a multivalent way, specificity can be lost as low affinity interactions gain avidity. In addition, there is always a concern that the transfer or attachment of a peptide or aptamer to a new molecule or particle may alter its conformation and binding affinity for the target of interest. Thus for targeting complex targets like cells where specificity is a greater concern than raw affinity, it would be ideal to select the optimal ligand in the very context in which it will be used. In one embodiment of the invention a nanoparticle library is generated, said nanoparticle library composed of DNA nanoparticles. To generate such a nanoparticle library, the invention teaches the use of rolling circle amplification of circular oligonucleotide templates to produce libraries of single stranded DNA nanoparticles that can be selected for binding to immunological checkpoint molecules. To generate said DNA nanoparticle library random nucleotide sequences are introduced in the template oligonucleotide, libraries can be produced and desired functions selected.

In some embodiments multimodal DNA nanoparticles can be created that specifically bind to cancer cells. In more embodiments, methods to optimize the creation of multimodal DNA nanoparticles are described. The desired particles are “bred” by a novel iterative selection and re-assortment method to create modular DNA nanoparticles that contain multiple distinct recognition elements.

In one embodiment of the invention, a method superior to aptamer technology is disclosed for generation of nucleic acids capable of selectively binding immunological inhibitory molecules. Although in some ways this technology is similar to aptamer technology in that it uses nucleic acid libraries as the basis for molecular recognition, it differs in several important ways. Each particle contains many copies of the sequence elements so there is intrinsic multivalent display of the modules, allowing avidity to compensate for low monovalent affinity. The modular nature of the particle template construction allows multiple distinct recognition elements to be assembled into a single molecular entity. Furthermore, the combinatorial selection method allows the optimal particle with multiple recognition elements to be evolved in the same molecular context in which it will be used, rather than grafting them on to some other framework or particle for application. The combinatorial method also adds an element of true molecular evolution in which novel combinations of modules can be created by re-assortment, akin to recombination in meiosis.

The invention disclosed combines the power of random libraries and biopanning selections, with molecular breeding concepts to create multifunctional molecules for cell binding. In addition to the conceptual advantages of this approach, discussed herein, once a particle has been selected and sequenced, other laboratories can easily create that particle from bacteria containing the cloned sequence or a synthetic oligonucleotide and a few simple molecular biology steps. The RCA reaction is scale-able and far less complicated than hybridoma technology. The pioneering approach of creating a single molecule nanoparticle with modular functionality is groundbreaking in its flexibility and potential for development as a platform for applications beyond just those discussed in detail here. Some embodiments provided herein are unique and innovative in at least three major ways. First, the module designs are unlike any other library format in flexibility and ease of implementation. Second, selection formats are the same as the application format meaning the selected particles can be used immediately without the need to chemically alter or conjugate them to another molecule or particle. Finally, compared to other nanoparticle materials, DNA is non-toxic and antisense oligonucleotides, aptamers, gene therapy, and CpG oligonucleotides have all been used in human trials.

In one embodiment DNA nanoparticles are produced by enzymatic DNA synthesis using a strand displacing DNA polymerase, phi29, and a circular oligonucleotide template. The oligonucleotide circle is typically produced by ligation of a 100-200 base pair linear oligonucleotide with a short (30 bp) oligonucleotide complementary to the ends. The ligation oligonucleotide also serves as the initiating primer for the RCA reaction. Phi29 polymerase is highly processive (.about.70 kb) and produces a linear increase in single stranded DNA for over an hour in a typical reaction. The resulting RCA products are concatemers complementary to the template circular oligonucleotide. These long single stranded products collapse into randomly coiled nanoparticles, a property that has been exploited for counting individual RCA events. The size of the particles is a function of the time and efficiency of the RCA reaction. The reaction can be stopped by the addition of EDTA or heat inactivation of the phi29 polymerase, though the latter may lead to aggregation of the DNA particles. The particles can be visualized with either single stranded or double stranded fluorescent DNA binding dyes due to the double stranded character that results from internal base pairing. For analytical purposes the particles can be made fluorescent by the inclusion of fluorescently labeled nucleotides during the synthesis. Alternately a fluorescently labeled oligonucleotide probe can by hybridized to the particles.

Dynamic Light Scattering (DLS) is a common technique for measuring the properties of nanoparticles such as size and zeta potential. DLS uses the time autocorrelation of a signal of scattered light to determine the polydispersity and average diffusion coefficient, which through the Stokes-Einstein equation is related to the average dynamic radius. RCA reactions were carried out for four time points (10, 30, 45, 60 minutes) and were stopped by the heat inactivation of the polymerase at 65° C. for 10 minutes. The samples are then immediately measured by DLS. For a monodisperse sample the autocorrelation plots should show a single exponential decay, the exponent coefficient of which is known as the first moment and is used to calculate a Z-average size. The second moment is used to calculate the deviation from monodisperse and is known as the polydispersity index (PdI), which is a measure of relative peak width of the Gaussian size distribution. In general if the PdI is greater than 0.25 it is recommended to use a secondary algorithm called Non-Negative Least Squares (NNLS) which models the autocorrelation curve as a contribution of several size samples and extracts individual peak data.

In one embodiment of the invention a library of DNA nanoparticles is generated from a template oligonucleotide that has a random stretch of bases in the middle, flanked by PCR primer sites. These ends also bind to the ligation oligonucleotide to circularize the template for RCA amplification. The random sequence is 60 nucleotides long and is flanked by defined sequences that can be used to hybridize fluorescent or otherwise labeled probes for visualization or purification. Using this design about 10 billion unique DNA nanoparticles can be produced in a small volume (e.g., 50 mu l) RCA reaction. The particles are screened for the desired binding activity and the binders amplified by PCR using the Stoffel fragment of Taq polymerase that lacks 5′ to 3′ exonuclease activity. The use of Stoffel fragment greatly increased the amplification efficiency, presumably due to the concatemeric nature of the DNA nanoparticles. Following amplification several rounds of asymmetric PCR are performed to increase the copy number of the template strand that is then circularized by ligation and subjected to RCA to regenerate the DNA particles. The cycle of screening, amplification, and particle regeneration is repeated for several (e.g., 5-10) rounds.

In one embodiment of the invention, the DNA nanoparticles are stable over the time of the experiments. However, the particles are fairly resistant to exonuclease and endonuclease degradation. Since the particles are formed from a continuous single stranded DNA molecule, each particle has only one 3′ and one 5′ end which may account for their resistance to exonuclease digestion. Most endonucleases, including DNAse, prefer double stranded DNA as a substrate. The nuclease resistance could increase by polymerizing nucleotides with altered backbone chemistries. The phi29 polymerase can incorporate phosphorothioate backbone nucleotides, although the rate of polymerization is marginally slower. Furthermore, some embodiments provided herein include methods and compositions to develop multimodal particles that bind to a target cell type through combinatorial “breeding” of DNA nanoparticles. The overall strategy is to first optimize the selection methodology, then apply the method to a panel of cell lines from each of two cancer types and confirm their usefulness in several cell binding applications. The rationale for using multiple cell lines from a couple of cancer types is to allow the selection of particles that are tumor specific but not cell line specific and to then be able to cross compare. If necessary, subtractive screening methodologies are employed to prevent the recovery of non-specific cell binding particles. Cell lines or normal lines (e.g. NIH-3T3) are used for subtraction.

In one embodiment of the invention, libraries are ligated as follows: the template oligonucleotide is mixed with the ligation primer in T4 ligase buffer to final concentrations of 100 nM and 300 nM respectively. The mixture is heated to 95° C. and allowed to cool slowly to room temperature. T4 ligase is added and the reaction incubated for 1 hour at 37° C. For multimodal libraries, there are multiple ligation primers, and all but one primer are dideoxy terminated so that each multimodal template circle is primed at only one location. Subsequently, the ligation mixture is added to an RCA reaction mix containing phi29 polymerase. The final concentration of the ligated template oligonucleotides is 1 nM, meaning that in a 100 mu l reaction about 6×10¹⁰ DNA nanoparticles are created. When the initial library is made each of these particles will contain a unique sequence. The reaction proceeds for 30 minutes at 30° C. and is terminated with EDTA. This produces particles .about 250 nm in size. The RCA reactions are monitored in real time with Oligreen, a single and double stranded DNA binding fluorescent dye, to confirm linear amplification. Since the amount of template DNA ligated and amplified is the same round to round, this rate should be constant and thus serves as a quality control checkpoint.

The selection step can be performed in several ways, depending on the application. In one embodiment, the particles are incubated with the immune suppressive ligand or receptor of choice. If non-specific binding is recovered, subtractive approaches can be used in which the library is first or concurrently counter selected against an irrelevant target to remove non-specific binding. This can be done by preincubating the library with the irrelevant target to absorb non-specific binding or, if the target and irrelevant target can be easily separated, the library can be added to a mixture of both with the target cells then later removed. Typical incubation times are 30 minutes to an hour at either room temperature or 37° C. 10⁵ cells are mixed with the entire RCA reaction from step 2. Step B. Washing is performed by centrifuging the cells and aspirating the liquid, then resuspending the cells and transferring to a new tube for the next wash. Three to five wash steps will be performed. Transferring to new tubes at each steps minimizes the recovery of plastic binding particles. Step C. Particle recovery. Since each particle is a concatemer of several hundred copies of the basic unit sequence, PCR amplification of single particles or even particle fragments is possible. Therefore, the particles can be recovered by lysing the washed cells followed by 1 hour treatment with proteinase K. The cell lysate is added to the PCR reaction in the next step, ensuring recovery of both external bound particles as well as particles that may have been internalized by the cells.

One aim of the amplification step is to regenerate the population of template oligonucleotides that can be ligated and used to generate a pool of particles reflective of the particles recovered in the selection step. The main amplification is by PCR and the desired template DNA strand is secondarily enriched over the complement by asymmetric PCR using only the primer for the desired strand. a) Stoffel PCR. PCR amplification of these DNA nanoparticles is much more efficient when the Stoffel fragment of Taq polymerase, which lacks 5′-3′ exonuclease activity, is used instead of conventional Taq polymerases, which has 5′-3′ exonuclease activity. This may be because of the concatameric nature of the DNA particle strand, which could bind many primers in the initial rounds of PCR. The extending polymerase would run into the primed strand downstream of it and begin digesting, leading to very inefficient polymerization from the particle strand. The PCR reactions are monitored in real time with Sybr green and stopped once the production of PCR product plateaus. The real time plots also allow quantitative estimates of the relative amount of DNA, and by inference the number of particles, recovered in each round. b) asymmetric PCR. The PCR product is diluted into a new reaction mixture that contains only the primer that will produce the ligatable template strand. This primer has a 5′ phosphate. The reaction is run for 10 cycles.

After a successful selection, candidate particles are further analyzed from the final pool. To obtain individual particles, the final pool are amplified by Stoffel PCR and cloned into a plasmid sequencing vector. Once cloned, 10-20 candidates are sequenced to determine the extent of sequence diversity in the final pool. Each candidate can be regenerated by PCR/asymmetric PCR amplification from the plasmid. The design of each random module can be subject to some constraints. The minimum length of an oligo that can effectively circularize is reported to be around 80 bp. Since the flanking PCR/ligation primers sites are 30-40 bp, the randomized region can be at least about 40 bp. The potential diversity of any such library is much greater than the sample size. For example, a 60 by library has 4⁶⁰ different possible sequences, about 1×10³⁶. Since typically 10¹⁰-10¹¹ particles can be created in a reasonable volume, only a tiny fraction is sampled of the possible number, and any particular batch of the library is a unique subset of the possible with each individual sequence represented by a single molecule. However, 10¹⁰ particles will likely contain any given 19 by motif at least once and smaller motifs will be well represented within the sampled population.

The overall concept of the modular library screening method can be divided into a “panning” phase, where binders within the population are selectively enriched, and a “breeding” phase in which the multimodal particles are re-assorted in each cycle so that novel combinations can be generated and the optimal combinations enriched.

When multimodal particles are created there is an additional combinatorial element. If 3 libraries of the size above are randomly combined into multimodal particles, then there would be 10³⁰ different combinations, treating each module as a discrete entity. Since there may be limits to 10¹⁰ particles due to the physical constraints of particle synthesis, it may not be ideal to combine libraries in the first few rounds of selection. In fact, it is unlikely that any particular combination recovered in the first round would reform in the second while the remaining diversity is still high. On the other hand, if particular combinations of modules would be optimal together but are not particularly good on their own, then delaying combination of the library may result in the desirable modules disappearing from the population before they have a chance to team with the others for selective advantage. However, in the absence of a rigorous model of selection kinetics and fitness landscapes, an empirical approach can be used. Three selection schemes can be tried. In the first, the libraries are assembled into the multimodal format prior to the first round and in all subsequent rounds. However, for the first three rounds the particles are amplified as a single unit and the modules are not be re-assorted. Non-modular PCR ensures that any particular combination of modules that comes through the first round will be amplified to higher copy number before the re-assortment process begins. For subsequent rounds, the recovered particles are split and some amplified as a single unit by the non-modular method and the rest amplified as modules and recombined, ensuring representation of the selected combinations as well as generating new combinations of modules. The second selection scheme attempts to pre-enrich the modular pool for desired activity by screening the single component libraries for several rounds prior to combination in multimodal format. The individual libraries are screened for 5-10 rounds until there is evidence in each of enrichment for binding clones (indicated by an increased in the number of particles recovered in each round as determine quantification from the real time PCR amplification step). The combinatorial strategy is then be pursued for several rounds. The final strategy is a hybrid of the first two. Three to five rounds of selection are performed with each of the component libraries, and then the following rounds are done using only the combinatorial approach.

1. A method of producing a DNA nanoparticle possessing selectively binding ability to an immune inhibitory molecule comprising the steps of:
a) obtaining a circular single-stranded nucleic acid template;
b) contacting said circular single-stranded nucleic acid template with a nucleic acid polymerase; and
c) amplifying said template with said polymerase to produce said DNA nanoparticle in a manner such that said nanoparticle comprises a concatemer of said aptamer sequence.

2. The method of claim 1, wherein said nucleic acid polymerase is a strand displacing DNA polymerase.

3. The method of claim 2, wherein said strand displacing polymerase is selected from the group consisting of phi29 polymerase, Klenow fragment, VENT® (Exo) DNA polymerase, 9° Nm DNA polymerase, Bst DNA polymerase, M-MuLV reverse transcriptase, and AMV reverse transcriptase.

4. The method of claim 1, further comprising circularizing a linear nucleic acid template to produce said circular nucleic acid template.

5. The method of claim 1, wherein said template encodes an aptamer sequence;

6. The method of claim 1, wherein said DNA nanoparticles are selected for ability to bind to immune suppressive molecules by the steps of
a) generating a library of nanoparticles comprising putative aptamers;
b) contacting said library to a capture probe; and
c) selecting for a nanoparticle that binds said capture probe.

7. The method of claim 1, wherein said capture probe comprises an immune suppressive molecule.

8. The method of claim 7, wherein said immune inhibitory molecule is a checkpoint inhibitor.

9. The method of claim 7, wherein said immune inhibitory molecule is selected from a group of molecules comprising of:
a) PD-1;
b) PD-1L; and
c) CTLA-4.

10. The method of claim 1, wherein said DNA nanoparticles are utilized for immunization into an animal, subsequent to immunization, antibodies generated in response to said DNA nanoparticle immunization are extracted and said antibodies are utilized to immunize another animal, said animal generating an anti-idiotypic antibody.

11. The method of claim 10, wherein said anti-idiotypic antibody binds to an immune inhibitory molecule with higher affinity as compared to original DNA nanoparticle.
